(19) United States
(12) Patent Application Publication
(10) Pub. No.: US 2017/0224497 A1
(43) Pub. Date: Aug. 10, 2017

(54) Improved Implant Surface

(71) Applicant: Ossis Limited, Christchurch (NZ)

(72) Inventors: Madeleine Bess Martin, Christchurch (NZ); John Arthur Calder, Tauranga (NZ)

(21) Appl. No.: 15/500,524

(22) PCT Filed: Jul. 30, 2015
(86) PCT No.: PCT/NZ2015/050098

§ 371 Date: Jan. 30, 2017

(30) Foreign Application Priority Data
Jul. 31, 2014 (NZ) 628129
Jul. 31, 2014 (NZ) 628131

(51) Int. Cl. A61F 2/36; A61F 2/30

(52) U.S. Cl. CPC A61F 2/3676; A61F 2/3662; A61F 2/30767; A61F 2/30942; A61F 2002/30845; A61F 2002/30914; A61F 2240/002

(57) ABSTRACT

This invention relates to orthopaedic implants having one or more regions of three dimensional lattice that substantially replicate the trabecular orientation within a healthy bone from the same anatomical location as the implant. A method for the manufacture of such orthopaedic implants is also disclosed using additive manufacturing techniques and patient-specific anatomical information.

### CROSS-REFERENCE TO RELATED APPLICATIONS

This application is a U.S. National Stage Entry of PCT/NZ2015/050098 filed Jul. 30, 2015, which claims the benefit of priority to New Zealand Patent Application No. 628129 filed Jul. 31, 2014, and New Zealand Patent Application No. 628131 filed Jul. 31, 2014, each of which are incorporated in their entireties by reference.

### TECHNICAL FIELD

This invention relates to orthopaedic implants including an improved surface design. More particularly, the invention relates to orthopaedic implants having a three dimensional lattice surface with a specific orientation.

### BACKGROUND ART

One of the key factors in successful placement and acceptance of an orthopaedic implant is the osseointegration between a load-bearing implant and the existing patient bone.

Osseointegration involves the formation of a direct interface between an implant and bone without intervening soft tissue, resulting in a firmly positioned implant that is unlikely to cause any pain or problem to the patient. Effective osseointegration has been shown to be promoted by using implants with a porous metal construction.

One example of such implants are those formed with “Trabecular Metal™” technology developed by Zimmer as a structure for dental and orthopaedic implants. This trabecular metal provides an average pore size of 440 μm with a 3D tantalum lattice similar to cancellous bone. The structure of the “trabecular metal” is such that pore sizes are consistent across the entire implant. While this type of structure has been proven to increase osseointegration as well as soft tissue vascularisation, the consistent iterative nature of the surface is not able to closely correspond to specific features and orientations of cancellous bone and trabeculi within either specific patients or within specific bones in the body. This in turn limits the potential of the osseointegration that can occur, as one standard surface interface is not able to provide the best outcome for all patients or for all possible implant sites within the body.

Cancellous bone or trabecular bone is typically located at the proximal and distal ends of long bones and internally in other bones as well. It contains a greater surface area to mass ratio compared to compact bone as it is less dense. Trabecular bone is generally formed of rod shaped bone tissue and plate shaped bone tissue, and is oriented within bone to reflect the mechanical stresses placed on that bone. Over time, this orientation is formed specifically within each patient in response to the particular loads placed on their bones based on the way they move. Common patterns in trabecular orientation are also seen across patients in specific areas of the body. For example, the proximal femur of a healthy patient typically displays a range of different groups of trabecular bone oriented in specific ways, including the greater trochanter group, principle compressive group and principle tensile groups of trabecular bone located at positions across the proximal femur that reflect the direction of forces applied across the hip. Similar trabecular patterns can be seen in other load bearing bones in other regions of the body.

Known surfaces of orthopaedic implants with symmetrical lattices or three dimensional lattices do provide additional surface area to aid osseointegration, however they are not designed to transfer stress to surrounding bone based on the trabecular orientation or orientation of specific components within a surface mesh, scaffold or lattice on the implant surface.

This mismatch between the architecture of the currently available trabecular lattices and the disposition of the trabeculi of the host bone can lead to a weakening of the surrounding bone and reduce the ability to build strong bone in areas that will be subject to greater stresses.

### Object of the Invention

It is an object of the invention to provide an orthopaedic implant that will provide improved osseointegration between the implant and the bone and positively influence the strength, quality and quantity of host bone, together with a method for the manufacture of such an implant.

Alternatively, it is an object to provide an implant with a specific trabecular or surface lattice orientation.

Alternatively, it is an object of the invention to at least provide the public with a useful choice.

### SUMMARY OF THE INVENTION

According to a first aspect of the invention there is provided an orthopaedic implant, the implant including an outer surface having one or more regions of three dimensional lattice that substantially replicate the trabecular orientation within a healthy bone from the same anatomical location as the implant.

Preferably, the implant is a patient specific implant.

Preferably, the healthy bone is selected from the patient's own healthy bone from the same anatomical location, or a healthy bone as selected from a population model database of bone structure information.

Alternatively, the trabecular orientation of substantially the entire healthy bone is replicated in the implant at corresponding locations.

More preferably, the implant includes regions of three dimensional lattice that substantially replicate the trabecular orientation along the lines of tension and/or compression forces in healthy bone.

In preferred embodiments the implant is a femoral implant. More preferably, the implant is a femoral stem implant or a femoral stem shell implant.

Preferably, the implant is a femoral implant including one or more regions of three dimensional lattice, the lattice having with a three dimensional structure that substantially replicates or partially replicates the trabecular orientation along the tension and compression lines of stress of a femur.

In one preferred embodiment, the three dimensional lattice includes a plurality of plates and rods.

More preferably, the plates have a predetermined orientation with respect to the outer surface of the implant and the rods span adjacent plates to form a three dimensional lattice.

In preferred embodiments, the outer extremities or edges of the plates in the three dimensional lattice have at least one tapered edge.

Preferably, the implant includes a region of three dimensional lattice on a femoral implant wherein the plates having an oblique orientation with respect to the outer surface of the implant, with the outer extremities of the plates angled towards the distal end of the femoral implant.

More preferably, the plates are angled between 20° and 90° from the outer surface of the augment. Even more preferably, the plates are angled 30°-60° from the outer surface of the augment.

In preferred embodiments the three dimensional lattice has an elasticity of 5-25 GPa.

In further preferred embodiments, the implant is a femoral stem implant wherein the tensile strength of the femoral stem shell implant in a longitudinal direction is between 60-70 MPa.

In further preferred embodiments, the implant is a femoral stem implant wherein the compressive strength of the femoral stem shell implant in a longitudinal direction is 70-280 MPa.

According to a further aspect of the invention, there is provided an orthopaedic implant, wherein the outer surface of the implant includes one or more regions of three dimensional lattice extending from the outer surface of the implant, the three dimensional lattice including a plurality of plates and rods, wherein the plates have a predetermined orientation with respect to the outer surface of the implant and the rods span adjacent plates to form a three dimensional lattice.

In preferred embodiments of the invention, the orthopaedic implant is a patient-specific implant.

In preferred embodiments, the implant is a femoral implant. More preferably, the implant is a femoral stem implant or femoral stem shell implant.

In further preferred embodiments when the implant is a femoral stem implant or stem shell implant, the one or more regions of three dimensional lattice are located on the shaft of the femoral stem implant or on the outer body of the femoral stem shell implant.

Preferably, the plates have an oblique orientation with respect to the outer surface of the implant, with the outer extremities of the plates angled towards the distal end of the femoral implant.

More preferably, the plates are angled between 30° and 60° from the outer surface of the augment.

Preferably, the plates have a substantially quadrilateral or triangular shape.

Preferably, the rods are substantially elongate in shape.

In further preferred embodiments the plates forming the outer contour of the lattice comprise chiselled or tapered extremities or edges.

In preferred embodiments of the invention the outer surface of the implant comprises lattice of between 4-8 mm thickness.

Preferably, the lattice is formed with a range of differing thicknesses across the implant surface.

In further preferred embodiments of the invention the lattice and implant are integrally formed.

More preferably, the implant and lattice are integrally formed using additive manufacturing technology.

Preferably, the additive manufacturing method is electron beam melting (EBM) manufacturing or laser melting manufacturing.

In alternative embodiments, the lattice is applied to the implant following implant manufacture.

In further preferred embodiments of the invention the area of lattice further includes an antibacterial coating. This coating may be applied following manufacture of the implant, or included within the material used for integrally forming the implant during additive manufacturing.

More preferably, the antibacterial coating is an antibacterial coating including silver.

In preferred embodiments the three dimensional lattice has an elasticity of 5-25 GPa.

In further preferred embodiments, the tensile strength of the femoral implant in a longitudinal direction is between 60-70 MPa.

In further preferred embodiments, the compressive strength of the femoral implant in a longitudinal direction is 70-280 MPa.

Preferably, the three dimensional lattice and implant are integrally formed.

According to a further aspect of the invention there is provided a method for the manufacture of an orthopaedic implant, the implant including an outer surface having one or more regions of three dimensional lattice that substantially replicate the trabecular orientation within a healthy bone from the same anatomical location as the implant; the method including the steps of;
a) obtaining information regarding the trabecular orientation of healthy bone from the same anatomical location as the implant;
b) determining one or more patterns of trabecular orientation from step a) to be incorporated into the three dimensional lattice on the outer surface of the implant;
c) designing the implant to include the one or more patterns of trabecular orientation determined at step b); and
d) manufacturing the implant based on the design of step c) using additive manufacturing.

Preferably, the implant is a patient-specific implant and the method includes the steps of;
a) obtaining patient-specific information regarding a patient's bone geometry at a specific anatomical location;
b) obtaining information regarding the trabecular orientation of healthy bone from an equivalent anatomical location as in step a);
c) selecting one or more patterns of trabecular orientation from step b) to be incorporated into the three dimensional lattice on the outer surface of the implant;
d) designing the patient-specific implant based on the information obtained in step a), the design including one or more regions of three dimensional lattice having a pattern of trabecular orientation determined in step c); and
e) manufacturing the implant based on the design of step d) using additive manufacturing.

Preferably, the healthy bone is selected from the patient's own healthy bone from the same anatomical location, or a healthy bone as selected from a population model database of bone structure information.

Preferably, information regarding trabecular orientation of healthy bone is sourced from CT, micro CT, X-ray, multi-energy or MRI scanning methods.

Preferably, the step of determining the trabecular orientation occurs at two or more specific locations throughout the healthy bone.

More preferably, the trabecular orientation is determined along the lines of tension and compression forces in healthy bone.

In alternative embodiments, the trabecular orientation may include regions of longitudinal and obliquely oriented trabeculi to improve fixation of the implant to bone.

Alternatively, the trabecular orientation of the entire healthy bone is replicated in the implant at corresponding locations.

In one preferred embodiment, the three dimensional lattice formed by the method includes a plurality of plates and rods.

More preferably, the plates have a predetermined orientation with respect to the outer surface of the implant and the rods span adjacent plates to form a three dimensional lattice.

In preferred embodiments, the method includes the step of forming the outer extremities or edges of the plates in the three dimensional lattice with at least one tapered edge.

Preferably, the implant formed by the method is a femoral implant. More preferably, the implant is a femoral stem implant or a femoral stem shell implant.

Preferably, the method includes the step of forming a region of three dimensional lattice on a femoral implant wherein the plates having an oblique orientation with respect to the outer surface of the implant, with the outer extremities of the plates angled towards the distal end of the femoral implant.

More preferably, the method includes the step of angling the plates between 20° and 90° from the outer surface of the augment. Even more preferably, the plates are angled 30°-60° from the outer surface of the augment.

In one preferred embodiment, the implant is a femoral stem shell implant including an outer surface and an inner surface defining an internal area, the internal area including compressible or deformable material adapted to receive a femoral stem implant.

In alternative embodiments, the inner surface includes one or more attachment means adapted to engage or interlock with an inserted femoral stem.

Preferably, the three dimensional lattice and implant are integrally formed.

Preferably, the method includes the further step of application of an antibacterial coating, preferably an ion beam silver sputter coating.

Preferably, the additive manufacturing method is electron beam melting (EBM) manufacturing or laser melting manufacturing.

According to a fourth aspect of the invention there is provided a method for revision of a femoral stem, the method including the steps of;
a) inserting a hollow femoral stem shell implant as described in further detail above;
b) positioning a femoral stem implant within the stem shell implant of a) in a correct orientation based on a pre-operative planning;
c) securing the femoral stem implant into the femoral stem implant using cement.

According to a further aspect of the invention there is provided a method for enhancing osseointegration between patient bone and an implant surface, the method including insertion of an orthopaedic implant, wherein the outer surface of the implant includes one or more regions of three dimensional lattice extending from the outer surface of the implant, the three dimensional lattice including a plurality of plates and rods, wherein the plates have a predetermined orientation with respect to the outer surface of the implant and the rods span adjacent plates to form a three dimensional lattice.

According to a further aspect of the invention there is provided a method for enhancing osseointegration between patient bone and an implant surface, the method including insertion of an orthopaedic implant including an outer surface and an inner surface, wherein the outer surface includes one or more regions of three dimensional lattice with a three dimensional structure that substantially replicate the trabecular orientation of healthy bone from the same anatomical location.

According to a further aspect of the invention there is provided a kit of parts for use in a femoral revision, the kit including the femoral shell implant as described in more detail above, together with a femoral stem adapted to be received within the orthopaedic implant.

For the purposes of this invention the term “plate” should be taken to mean a substantially flat form having a first surface and opposing second surface. One example of this is shown in more detail in the accompanying figures.

The “rods” of the present invention are substantially elongate in form and the term “rod” should not be limited to a particular cross-sectional shape, but refer to any shape that is elongate in form.

The term “shell” should be interpreted to mean any implant that is capable of receiving a further orthopaedic implant, thereby creating a “shell” around the implant being received. While the shell may be hollow, it may also have a porous or deformable core that allows a further orthopaedic member such as a femoral stem to be inserted.

Further aspects of the invention, which should be considered in all its novel aspects, will become apparent to those skilled in the art upon reading of the following description which provides at least one example of the implants of the current invention.

### DESCRIPTION OF THE DRAWINGS

One or more embodiments of the invention will be described below by way of example only, and without intending to be limiting, with reference to the following drawings, in which:

FIG. 1 shows a cross-sectional proximal femur of a healthy patient showing an example of trabecular orientation within the cancellous bone;

FIG. 2 shows a further representation of typical trabecular orientation within the femur;

FIG. 3 shows a close up (not to scale) view of the rods and plates of specifically orientated trabeculi on an implant in one embodiment of the invention;

FIG. 4 shows a further close up of a cross section of trabeculi on the outer surface of an implant with plates and rods forming a three dimensional lattice in one embodiment of the invention;

FIG. 5 shows a perspective view of the plates and rods of the three dimensional lattice in one embodiment of the present invention;

FIG. 6 shows a cross section of a proximal femur (without head and neck portion) including a femoral stem shell implant in one preferred embodiment of the invention;

FIG. 7A shows a close up of the trabecular orientation as shown in FIG. 2;

FIG. 7B shows a femoral stem shell implant including specific trabecular orientation matching the trabecular orientation shown in FIG. 7A in one embodiment of the invention; and

FIG. 8 shows a femoral stem augment including three dimensional lattice on the outer surface of the implant in one embodiment of the invention.

### DETAILED DESCRIPTION OF THE INVENTION

The orthopaedic implants and methods of the present invention are designed to increase osseointegration with patient bone and cause accretion and strengthening of the existing bone by providing an implant that incorporates surface features that mimic those of healthy bone and/or specific features within a patient bone at a similar location in the body.

The success of the osseointegration may be improved by more closely replicating the trabecular orientation that would be present in a corresponding healthy bone.

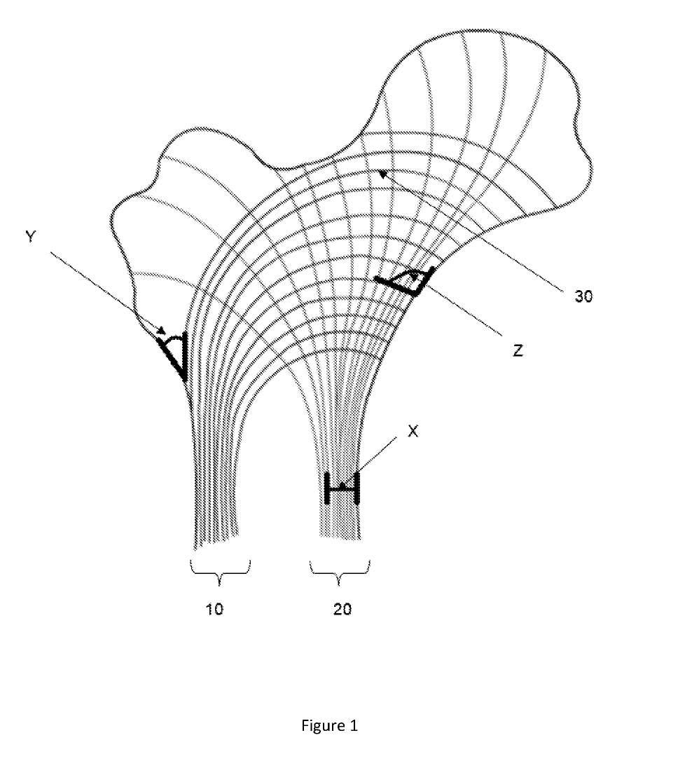

Load bearing bones such as the femur are subject to tensile and compressive stresses. These stresses result in the formation of particular patterns within the cancellous bone in order to optimise the bone's ability to absorb these stresses. Trabecular orientation is important for allowing stress transfer in a range of bones within the anatomy. For example, trabecular orientation within healthy cancellous bone of the femur, as shown in FIG. 1, can be seen to follow curved paths from the one side of the femur shaft, radiating outwards towards the opposite of the side of the femoral head.

Curved paths shown by group 10 correspond to the path of tension stress within the femur and as a result, corresponding trabecular orientation. Paths 20 correspond to the path of compression stress and the corresponding trabecular orientation. The stress lines cross each other at substantially right angles 30, further increasing the strength of the cancellous bone structure.

Trabecular orientation is important for allowing stress transfer in a range of bones within the anatomy. For example, trabecular orientation within the tibia or radius also play a vital role in absorbing stresses within the bone. The implants of the present invention may be adapted for use in a variety of locations where trabecular orientation is important, such as the tibia or fibula in the lower leg, or the humerus, pelvis, skull, mandible, ulna or radius in the arm.

Incorporating specific trabecular orientation within the surface three dimensional lattice of an implant reduces the risk of stress shielding of the surrounding bone. If the implant is does not effectively transfer load to the surrounding tissue, osseointegration is not encouraged, resulting in patient pain, loosening of the implant or eventually shortening of the limb (for example the leg as the implant subsides into the femur).

The structure and/or pattern of the lattice within the implant of the present invention is designed to improve osseointegration by providing an implant surface with a trabeculation pattern that mimics that of healthy bone, in particular trabecular orientation in the femur is exemplified. Re-creating this surface structure on an implant encourages the transfer of stress from the hip joint to the surrounding bone, stimulating osseoinduction, which in turn results in improved osseointegration of the implant with the surrounding femur.

The following description of the invention will be made using the femur and a femoral implants (either in the form of a femoral stem, or a femoral shell that is designed to receive a femoral stem) as an example, however this is not intending to be limiting. The implants and methods of the present invention may be applied at numerous regions throughout the body where improved osseointegration between an implant and patient bone is desired, as described above.

Trabecular structure within healthy bone is known to consist of a structure formed from plates and rods. The plates are wider, flatter more “plate like” parts of the cancellous bone, while rods are thinner, rod shaped elements of lower density that provide supporting structure between the plates.

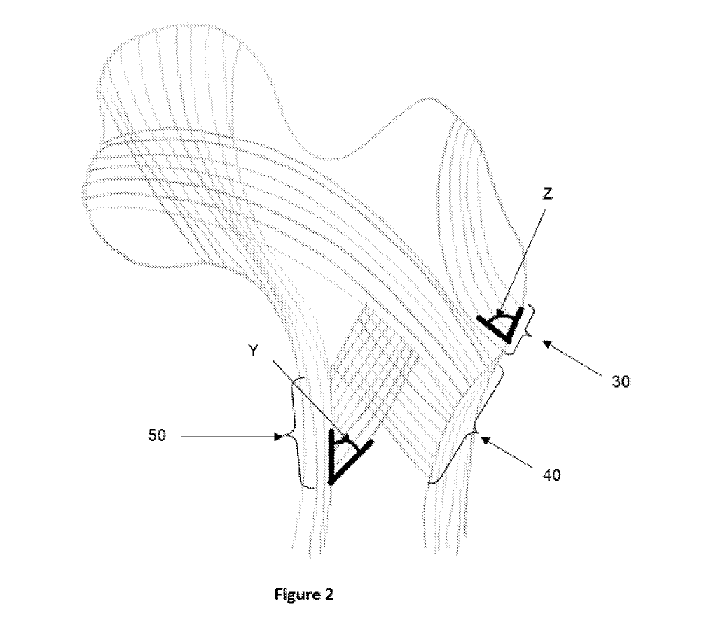

In one preferred embodiment of the invention, a femoral implant is provided that includes an outer surface including at least a portion of three dimensional lattice that mimics the trabecular orientation of lines of tension and compression found in a healthy femur, as shown in FIG. 1 and FIG. 2, or of a specific patient, based on information derived from patient scans and analysis.

FIGS. 1 and 2 are stylistic representations and should not be seen as limiting either in the exact location and orientation of the plates and rods, or the size or shape of the plates and rods. In the formation of the lattices of the present invention using additive manufacturing, the lattice may include plates that are substantially flat and “plate like”, but may have irregular boundaries, include curves or be curved, or may be substantially, square, rectangular, circular, oval hexagonal, octagonal, decahedron or dodecahedron or other geometric shape that would be suitable for forming the edge of a plate like structure. Likewise, the rods are substantially elongate in shape in order to provide supporting struts between adjacent plates, but may be formed with a variety of different cross-sections, for example, but not limited to substantially, square, rectangular, circular, oval, hexagonal, octagonal, decahedron or dodecahedron.

The orientation of the plates within trabecular bone can be categorised by three orientations; transverse orientation, which indicates horizontal alignment, oblique orientation, indicating oblique alignment and longitudinal orientation which indicates vertical alignment. These terms are known in the art and are indicated in FIG. 1 by reference points X, Y and Z. The orientations depicted in FIG. 1 are not intended to be in anyway limiting, but are purely illustrative examples for the discussion of how trabecular orientation may be described with respect to different parts of a bone and to more clearly explain the preferred embodiments of the invention. Trabecular orientation may differ from patient to patient, particularly if specific stresses have been placed upon bone that may result in differences in trabecular orientation.

Reference point X shows an example of transverse orientation of trabeculi with respect to the femoral shaft. The trabeculi are oriented with the majority of the plates' surface area substantially horizontal to the shaft wall, held together by cooperating rods. Point Y shows an oblique orientation, with trabecular plates aligned at an approximately 45° angle with respect to the bone wall and point Z shows a longitudinal orientation of the trabecular plates with respect to the bone wall at point Z.

FIG. 2 shows a further example of trabecular orientation within the femur, including additional trabecular orientation areas 30, 40 and 50. As with FIG. 1, the orientation in these areas can be defined as oblique, transverse or longitudinal, with essentially oblique orientation Y within areas 40 and 50 and longitudinal orientation in group 30 indicated by reference Z.

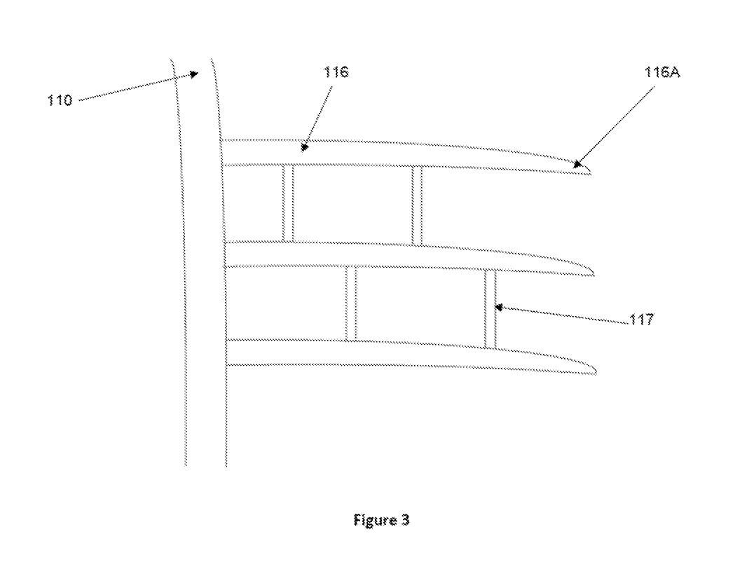

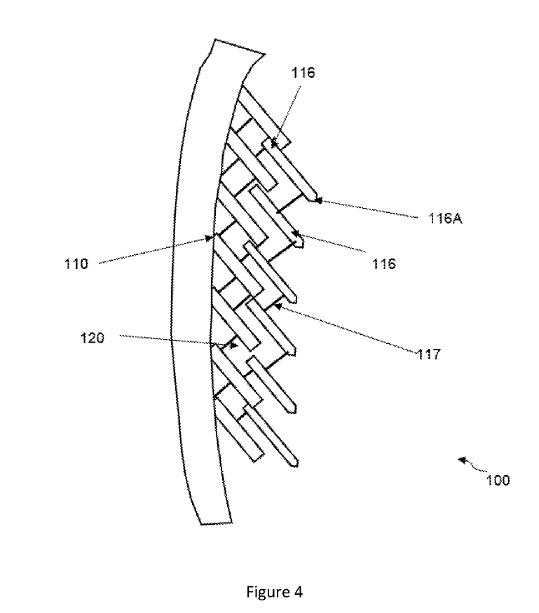

FIGS. 3 and 4 show a basic view of a lattice structure 100 formed from plates 116 and rods 117. In this embodiment, plates 116 have a substantially longitudinal orientation with the plates orientated at approximately 90° from the outer surface of the implant wall 110. Rods 117 are located between adjacent plates 116 to provide stability and structural integrity.

In FIG. 4, spaces 120 formed between rods 117 and plates 116 allow for ossification of new bone into lattice 100, providing strong osseointegration between the implant via lattice 100 and patient bone.

FIG. 4 shows plates 116 and rods 117 making up lattice 100, with the plates 116 having an oblique orientation, with the plates angled at approximately 45° from the outer surface of implant 110. Preferably the lattice located on or formed with outer surface 110 is between 4 mm-8 mm thick, with increasing or decreasing plates and rods forming thinner or thicker areas of lattice as required for the specific anatomical area where the implant is being used. It may be desirable in patient-specific implants to increase or decrease the thickness of the lattice in order to provide an optimum outer contour that replicates the contours of patient bone, to fill voids or to contour around ridges.

In one embodiment of the invention plates 116 include a tapered or chiselled end 116A. This tapered end improves the interference fit between the edge of the lattice and the wall of patient bone, when placed in position. The tapered end is preferably angled such that once inserted, it digs in to the surrounding bone, providing a further anchor for the implant and preventing the shell from moving downwardly as pressure is applied from the hip region.

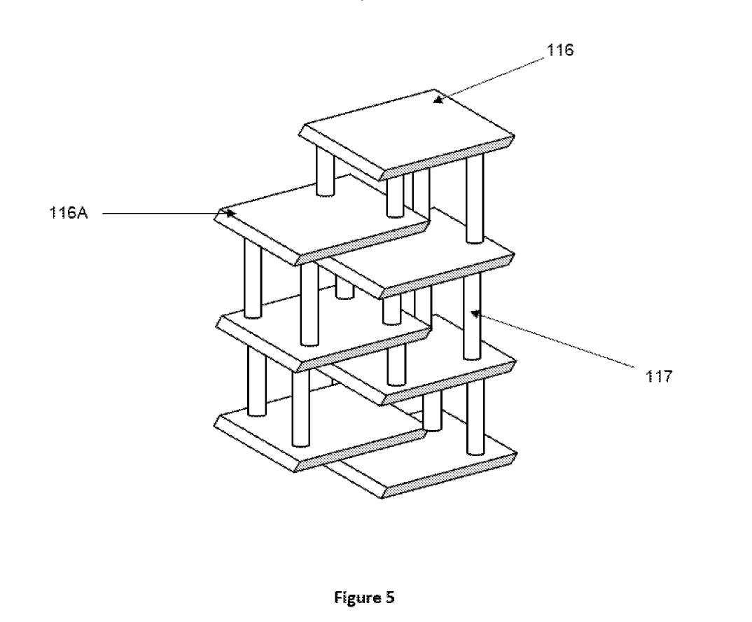

FIG. 5 shows a three dimensional example of plates 116 and rods 117 of the lattice 100 in one embodiment of the invention. In this embodiment, plates 116 are separated and supported by rods 117, with tapered plate ends 116A. The three dimensional arrangement of plates and rods depicted in FIG. 5 is one option for the arrangement of the plates and rods, however various arrangements may be employed as required to effectively arrange the lattice across curved and contoured surfaces and this should not be seen as limiting.

In preferred embodiments the lattice has an elasticity of 5-25 GPa which closely approximates the elasticity of healthy bone. This range is a guideline only and as would be understood by a person skilled in the art, the implant of the present invention will conform to ISO requirements. If these requirements change then the above ranges may also change to accommodate this.

It is envisaged that the lattice of the current invention may have a unit cell size of between 0.5-3 mm2, wherein a unit cell is regarded as the volume enclosed by 4 rods and 2 plates in the case of a substantially quadrilateral plates, or two plates and three rods in when the plates are substantially triangular in shape. It should be understood that this is not intended to limit the shapes of the plates, but clarifies the general meaning of “unit cell” for the purposes of this invention. In some cases the unit cells across a three dimensional lattice will not be regular, therefore the shape of unit cells within a single implant may also alter. Preferably, unit cells of this range result in a porosity of between 60-90%.

Plates 116 may include tapered or chiselled edges 116A at the extremities of the lattice. Such tapering or chiselling of the outermost edges of lattice 100 provides a decreased surface area allowing the implant to “dig in” to the surrounding bone once in position, further strengthening the bond between the implant and the patient tissue as discussed above.

The femoral stem shell implant of the embodiment of the invention described herein is designed to be used in either a femoral stem revision procedure, or primary femoral stem replacement, typically as part of a total hip replacement procedure where the bone is deficient or in other ways abnormal. In a typical insertion of a femoral stem, a stem implant is inserted directly into the patient's femur. Such femoral stems typically come in a range of standard shapes and sizes, with the most appropriate size selected for any given patient. In revision procedures, a typical insertion method is to force the implant stem further down the shaft of the femur into bone of better quality. The main disadvantage of this is that as load is transferred directly to the lower femur, osseointegration at the proximal femur is compromised. In order to overcome the problem of stress shielding the femoral stem shell implant of the present invention is designed to be inserted into the proximal shaft of the patient's femur, transferring load through regions of specific trabecular orientation within the surface lattice to the surrounding bone tissue, which encourages osseoinduction to heal and grow bone around the implant.

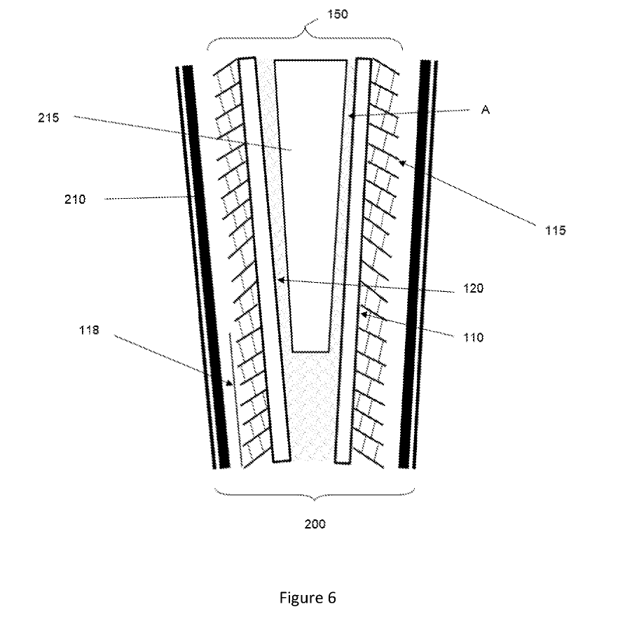

A cross-section of a femoral stem shell implant 150 in one embodiment of the invention can be seen in FIG. 6. FIG. 6 shows the existing cortical bone 210 of a patient's femoral stem 200. Stem shell 150 is designed to fit inside the femur, following removal of the femoral head and neck. This removal may include extraction of a pre-existing implant in the case of a revision, or removal of a diseased or damaged femoral head and neck as part of a primary replacement.

When a femoral stem is removed following a revision, the final state of the femur following extraction is often a hollowed out, thin shell of bone, often with fragments missing and usually weakened. The medial side of the neck and upper shaft may also be missing and sometimes the greater trochanter may have broken away or have been removed in an extended trochanteric osteotomy procedure so that residual cement from prior surgery may be removed.

Stem shell 150 is designed to be inserted into femoral stem 200 and includes an outer surface 110 and inner surface 120. Once in position, stem shell 150 can receive a corresponding femoral stem implant within region A, which may be a cavity defined by inner walls 120.

Cavity A is designed to receive a femoral stem implant 215, allowing it to be fixed in position using bone cement or similar. In alternative embodiment, region A may include a number of smaller cavities into which may be deformed into a single cavity when an implant is insert, or formed from a compressible or deformable material that may allow the insertion of a femoral implant by force, but will provide further structure and stability to the implant by filling any voids that may be present in region A following insertion of a femoral stem.

In preferred embodiments the stem shell implant is formed by the titanium alloy Ti6Al4V. Inner surface 120 is preferably formed with a slightly roughened texture to improve adherence of cement that may be used to secure a femoral stem within cavity A. In other embodiments inner surface 120 may be smooth, grooved, or polished and/or textured in different areas as may be required depending on the attachment means desired or type of femoral stem used. Surface 120 may also include attachment means that are capable of engaging or interlocking with an incoming femoral stem to improve stability or provide temporary support until more permanent attachment means are secured.

Outer surface 110 includes one or more regions of three dimensional lattice 100 with specific plate orientation, in relation to outer implant surface 110. In preferred embodiments of the invention the outer surface 110 of the stem shell augment may be entirely covered with the three dimensional lattice of the current invention, or specific areas will include the specifically oriented lattice. When used in patient specific augments, the placement and orientation of the lattice may be decided based on various types of information regarding the patient's anatomy. This may include information such as the location of a patient's best bone stock that will most successfully osseointegrate with the lattice, or avoiding areas of damage or specific anatomical tissue such as nerves, tendons, ligaments or blood vessels for example.

The implant 150 of FIG. 6 shows plates oriented approximately 60° from the outer surface 110 of implant 150, with the edges or extremities of the plates 116A angled downwards towards the distal end of implant 150. Once placed into the patient's femoral cavity, the downward orientation of the lattice 100 encourages the outer plates 16A to dig in to the edges of the surrounding tissue, firmly locating the implant in the correct position. Once the patient is mobile following the surgery, pressure and stress on the implant transferred from the pelvis will be transferred through the implant and lattice 100 to the surrounding bone, encouraging further bone growth. Stress on the implant from the hip and pelvis region will further encourage the tapered plate edges 116A to embed into the surrounding tissue. This is a significant advantage over other known implants with flat implant surfaces, or implants with uniform trabecular mesh having no exposed, specifically oriented edges, which are less able to transfer stress to the surrounding bone and may also shift in position (often further down the femur) once stresses are applied over a period of time.

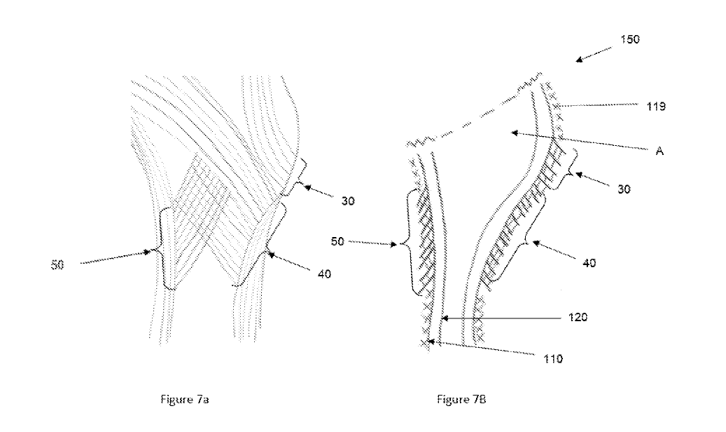

Outer surface 110 includes one or more regions of three dimensional lattice including areas of specific trabecular orientation, as shown in more detail in FIGS. 7A and 7B.

FIG. 7A shows a close up of the trabecular orientation of FIG. 2 in the location where femoral stem shell implant 150 may sit. Different regions of trabecular orientation are indicated by groups 30, 40 and 50. This trabecular orientation is shown transposed onto femoral stem shell implant 150 in FIG. 7B, where three dimensional lattice 115 are located on outer surface 110.

In preferred embodiments the three dimensional lattice has an elasticity of 5-25 GPa which closely approximates the elasticity of healthy bone. The tensile strength of the femoral stem shell implant in a longitudinal direction is between 60-70 MPa and the compressive strength of the femoral stem shell implant in a longitudinal direction is 70-280 MPa. These ranges are guidelines only and as would be understood by a person skilled in the art, the implant of the present invention will conform to ISO requirements. If these requirements change then the above ranges may also change to accommodate this.

In FIG. 7B, areas of three dimensional lattice with specific trabecular orientation 30, 40 and 50 are designed based on the orientation found in healthy bone shown in FIG. 7A, then replicated on the surface of implant 150. Plates 116 in regions 30, 40 and 50 are angled to mimic the trabecular orientation shown in 7A and are held together by rods 117.

Areas of outer surface 110 not required to be covered by lattice with a specific trabecular direction may have a standard uniform lattice surface. The lattice on these regions of outer surface 110 may be a three dimensional modified dodecahedron structure with an individual unit cell volume of between 0.5-3 mm, with a preferred volume of between 1.0 mm and 2.0 mm, for example.

Preferably the three dimensional lattice located on or formed with outer surface 110 is between 4 mm-8 mm thick. This is not intended to be limiting however, as it may be desirable in patient-specific implants to increase or decrease the thickness of the three dimensional lattice in order to provide an optimum outer contour that replicates the contours of patient bone.

The implants of the present invention may be formed in a custom shape or in preferred embodiments is formed as a patient-specific implant.

Patient-specific implants are known in the art and have a range of known advantages, the most obvious being that a patient-specific implant can be designed to optimally fit a patient's anatomy, resulting in greater success rates in orthopaedic replacement procedures. In the present invention a patient-specific femoral stem shell implant may be designed using data from CT-scans, X-rays, MRI scans, or radiography techniques for example.

Using the femoral stem shell implant as an example, when designing the optimum shape of the shell, information regarding the location of healthy bone, voids and areas of weakness can all be considered when designing the shape of outer walls 110 and the outer contours 118 of the areas of lattice 100.

As discussed above, the lattice depth on the outer wall 110 of implant 150 may be 4-8 mm. If the recipient femoral shaft includes regions where the bone is thinner, or has specific deformations, lattice 100 may be formed at varying depths in order to effectively fill any depressions to optimise contact between the lattice and bone tissue surface. In areas where voids may be located, lattice 100 may extend or be extendable into the voids in order to connect with bone graft that may potentially be packed behind.

As would be clear from the above description and FIGS. 3-5 exemplifying possible three dimensional lattices of the present invention, the regions of specific plate orientation may not extend across the entire surface of the implant. In regions where specific plate orientation is not required, outer surface 110 may be formed with a roughened surface, or may include a uniform lattice structure, (which may or may not be formed with a rod and plate structure) that mimics standard trabecular bone structure without the specific orientation.

Roughened surfaces may be produced using additive manufacturing to the exact required surface roughness, or alternatively surfaces may be sanded or altered post-manufacturing to achieve the exact surface required. Preferably the roughened surface has an Ra value of between 1-20 μm, with the higher Ra values indicating a rougher surface.

The preferred uniform lattice structure for use in the present invention (in areas where the lattice with plates and rods of specific orientation is not used) may be a three dimensional modified octagonal, decagon or dodecahedron structure with an individual unit cell structure of between 1 mm and 3.0 mm. Maintaining unit cell size is important to increase the chances of effective osseointegration with the implant. If unit cell size is too small, the lattice becomes difficult to clean and sterilise prior to use. If the unit cell size becomes too large, the structure loses mechanical integrity and osseointegration may also not be achieved as readily with a more spacious lattice structure. Other lattice structures may also be used on surface 110 as necessary and the above examples should not be seen as limiting.

The method of the present invention enables the design and manufacture of an orthopaedic implant having at least a region of three dimensional lattice that substantially replicates the trabecular orientation of healthy bone from the same anatomical region. This method allows formation of both custom (patient specific) and non-custom implants and can be applied using a range of different mesh types, including the example of the plates and rod type mesh described herein.

To manufacture and design a non-custom implant, information is obtained regarding the optimum trabecular orientation at the implant site. This information may be sourced from a population database of bone information generated from known scanning techniques and may be suitable for use across a wide range of patients with specific common characteristics, for example age, gender or levels of activity.

In the example of a femoral stem implant, the implant shape is selected and the one or more regions of specific trabecular orientation is overlaid onto the implant shape using a CAD design program for example. This step in the design process includes selecting the type of mesh to be used and angling, stretching or modifying the mesh such that the lines of trabeculation mimic those of the healthy bone.

If using a plate and rod type mesh, this step will including angling the plates with respect to the outer surface of the implants such that they lie either longitudinally, obliquely or transversely (for example) with the outer implant surface. When other mesh forms are used, this may include stretching a portion of the mesh design in a particular direction, or altering the thickness or spacing of the mesh at particular regions to mimic the trabeculation patterns of healthy bone.

For custom or patient-specific implants, instead of selecting a standard implant shape for use across a range of patients, the desired anatomical shape of the implant is determined using analysis of scanned imagery from a specific patient to optimise the contact between the shell and bone when located within a patient. Once the optimal shape of the implant has been determined, the design and placement of the three dimensional lattice can continue as described above.

In a patient-specific implant the placement of regions of specific lattice orientation on patient-specific implants may itself be patient-specific. This may not necessarily mimic the trabecular orientation that may be seen in the patient-specific imaging (this may be matched to a healthy bone from a database), but may also be designed to optimise fixation strength or to include additional regions of structure where they are required based on a range of factors as decided by a specialist. However, if a healthy specimen of the same bone is available in the same patient (for example the opposite femur), this may be used as the template for the design and placement of the trabecular orientation as well.

Once the required measurements and analysis of specific requirements from the orthopaedic specialist have been conducted (if required), a 3D model is developed. The model is then used to manufacture the patient-specific implant using additive manufacturing techniques, preferably EBM manufacturing. Following manufacture of the implant, the implant is then surface finished if necessary, cleaned and sterilised (if required) before being provided to a hospital or surgical professional for use.

Also disclosed within this invention is an orthopaedic kit for use in a primary hip replacement surgery or femoral stem revision surgery. The kit includes a femoral stem shell implant, for example similar to that shown in FIG. 6, comprising a surface lattice having regions of specific trabecular orientation, together with a femoral stem implant to be received within the cavity A and attached to inner surface 120 of implant 150.

The femoral stem implant may be a custom implant, or a non-custom implant. The use of a non-custom implant together with the femoral stem shell implant of the present invention overcomes many of the disadvantages that occur when such a stem is directly inserted into a patient's femur. The femoral stem shell implant with specific three dimensional lattice orientation as discussed herein is designed to reduce stress shielding normally associated with the direct insertion of a stem implant into a femur and promotes successful osseointegration with the patient's existing bone.

The ability to use an off the shelf primary femoral stem with a patient-specific shell implant with specific trabecular orientation is a much more economical than using a revision femoral stem. The presence of the shell implant removes the need for using stems with porous coatings or specifically designed stems for improving osseointegration, and allows the use of conventional femoral stems that can be inserted within the shell implant using bone cement or other attachment mechanisms.

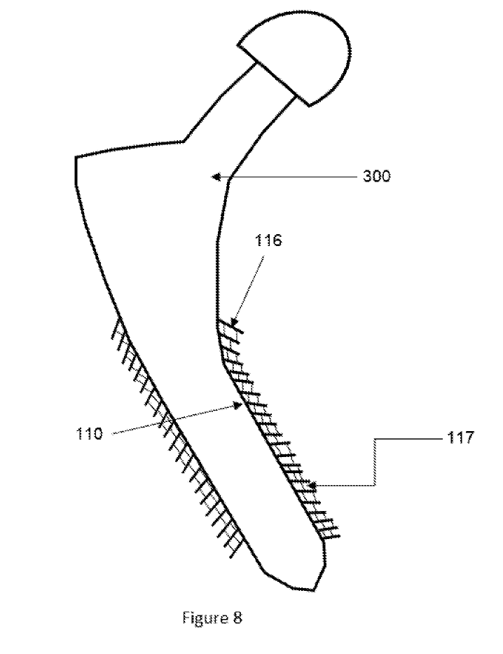

In alternative uses, the methods of the current invention may be applied directly to a femoral stem implant 300, as shown in FIG. 8. As with the femoral stem shell shown in FIG. 6, the femoral stem implant may include a three dimensional lattice of plates 116 and rods 117, the plates 116 angled obliquely, with the tapered ends of plates 116 pointing downward towards the distal femur, enabling better integration of the stem surface with surrounding bone. Femoral stem implant 300 may be used without a surrounding stem shell implant. In such cases the specific trabecular orientation aides in direct osseointegration between the stem and surrounding bone. The three dimensional lattice of the present invention may be applied to specific regions of the femoral stem augment , for example in between, or adjacent regions of cement. The method would allow the securing of the stem in position using cement, but also allow for regions of direct contact between the implant surface and surrounding bone, increasing osseointegration and improving stress transfer from the implant to the surrounding bone.

The present invention further allows for improved methods of enabling osseointegration between bone tissue and an implant surface. The presence of specifically oriented plates and rods within the lattice allows for, following the insertion of the implant, stress transfer to the weakened upper femur, thereby allowing the cortex to thicken up in response.

In a femoral stem and stem shell implants with a lattice with plates orientated obliquely on the implant surface as shown in FIGS. 5 and 6, such orientation will stop subsidence from occurring by engaging the residual cortex in using an interference fit. The trabecular orientation will also enable easier and more reliable re-attachment of an extended trochanteric osteotomy, by inhibiting any proximal migration of the greater trochanter that may be actuated by the pull of the attached gluteal muscles.

As mentioned above, the ability to provide specific orientation of plates and rods within a three dimensional lattice on the surface of an implant may be applied to implants at any number of sites throughout the body where such orientation aids in transferring stress effectively throughout the bone structure and/or promoting osseointegration. The above discussions in relation to a femoral stem shell implant are one of the many types of implants that may benefit from such technology but are not intended to be limiting.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

1. An orthopaedic implant, the implant including an outer surface having one or more regions of three dimensional lattice, the lattice including a plurality of plates and rods that substantially replicate the trabecular orientation within a healthy bone from the same anatomical location as the implant.

2. The implant of claim 1, wherein the implant is a patient specific implant.

3-4. (canceled)

5. The implant of claim 1, wherein the trabecular orientation of substantially the entire healthy bone is replicated in the implant at corresponding locations.

6. The implant of claim 1, wherein the implant includes regions of three dimensional lattice that substantially replicate the trabecular orientation along the lines of tension and/or compression forces in healthy bone.

7. (canceled)

8. The implant of claim 6, wherein the implant is a femoral stem implant or a femoral stem shell implant.

9. The implant of claim 8, wherein the implant is a femoral stem implant or femoral stem shell implant including one or more regions of three dimensional lattice, the lattice having with a three dimensional structure that substantially replicates or partially replicates the trabecular orientation along the tension and compression lines of stress of a femur.

10. (canceled)

11. The implant of claim 1, wherein the plates have a predetermined orientation with respect to the outer surface of the implant, the predetermined orientation based on the trabecular orientation of healthy bone at the same anatomical location.

12. The implant as claimed in claim 1, wherein the rods span adjacent plates to form a three dimensional lattice.

13. The implant of claim 1, wherein one or more of the plates in the three dimensional lattice have at least one tapered outer extremity or edge.

14. The implant as claimed of claim 13, wherein the implant is a femoral implant and includes a region of three dimensional lattice wherein the plates have an oblique orientation with respect to the outer surface of the implant, with the outer extremities of the plates angled towards the distal end of the femoral implant.

15-29. (canceled)

30. A method for the manufacture of an orthopaedic implant, the implant including an outer surface having one or more regions of three dimensional lattice, the lattice including a plurality of plates and rods, that substantially replicate the trabecular orientation within a healthy bone from the same anatomical location as the implant; the method including the steps of;
a) obtaining information regarding the trabecular orientation of healthy bone from the same anatomical location as the implant;
b) determining one or more patterns of trabecular orientation from step a) to be incorporated into the three dimensional lattice on the outer surface of the implant;
c) designing the implant to include the one or more patterns of trabecular orientation determined at step b); and
d) manufacturing the implant based on the design of step c) using additive manufacturing.

31. The method as claimed in claim 30, wherein the implant is a patient-specific implant and the method includes the steps of;
a) obtaining patient-specific information regarding a patient's bone geometry at a specific anatomical location;
b) obtaining information regarding the trabecular orientation of healthy bone from an equivalent anatomical location as in step a);
c) selecting one or more patterns of trabecular orientation from step b) to be incorporated into the three dimensional lattice on the outer surface of the implant;
d) designing the patient-specific implant based on the information obtained in step a), the design including one or more regions of three dimensional lattice having a pattern of trabecular orientation determined in step c); and
e) manufacturing the implant based on the design of step d) using additive manufacturing.

32. The method of claim 30, wherein the healthy bone is selected from a population model database of bone structure information.

33. The method of claim 30, wherein the healthy bone is selected from the patient's own healthy bone from the same anatomical location.

34-35. (canceled)

36. The method of claim 30, wherein the trabecular orientation is determined along the lines of tension and compression forces in healthy bone.

37. (canceled)

38. The method of claim 30, wherein the trabecular orientation of the entire healthy bone is replicated in the implant at corresponding locations.

39-47. (canceled)

48. The implant of claim 1, wherein the implant is a femoral stem shell implant including an outer surface and an inner surface defining an internal area, the internal area including compressible or deformable material adapted to receive a femoral stem implant.

49. The implant of claim 1, wherein the implant is a femoral stem shell implant including an outer surface and an inner surface defining an internal area, the inner surface including one or more attachment means adapted to engage or interlock with an interested femoral stem.

50-52. (canceled)

53. A method for enhancing osseointegration between patient bone and an implant surface, the method including insertion of an orthopaedic implant including an outer surface and an inner surface, wherein the outer surface includes one or more regions of three dimensional lattice with a three dimensional structure that substantially replicates the trabecular orientation of healthy bone from the same anatomical location.

54-55. (canceled)
